(19) United States
(12) Patent Application Publication
(10) Pub. No.: US 2005/0106273 A1
(43) Pub. Date: May 19, 2005

(54) Appetite suppressant

(76) Inventor: Gholam Peyman, New Orleans, LA (US)

Correspondence Address: WOOD, HERRON & EVANS, LLP, 2700 CAREW TOWER, 441 VINE STREET, CINCINNATI, OH, 45202, US

(21) Appl. No.: 10/744,269

(22) Filed: Dec. 22, 2003

Related U.S. Application Data
(60) Provisional application No. 60/510,857, filed on Oct. 14, 2003.

(51) Int. Cl. A61K 35/78

(52) U.S. Cl. 424/750

(57) ABSTRACT

A method of suppressing appetite is disclosed. An amount of wheat bran sufficient to suppress appetite is consumed prior to or with a meal to achieve satiety and desirable weight reduction. Postum®, a coffee-substitute and containing wheat bran as the quantitatively main ingredient, may be used in one embodiment. It may be prepared by dissolving in boiling water as a beverage or adding to a solid or semi-solid food. Consuming Postum® before or with a meal suppresses appetite so that less food is desired during the meal, yet the individual does not feel hungry. Postum® is a natural product and does not contain herbs, botanicals, stimulants, or synthetic components that may be harmful when consumed.

This application claims priority to U.S. Provisional Patent Application Ser. No. 60/510,857, filed Oct. 14, 2003.

### FIELD OF THE INVENTION

The invention is directed to a method of appetite suppression.

### BACKGROUND

A composition of wheat bran, wheat, molasses, corn-derived maltodextrin, and natural coffee flavor is commercially available under the trademark Postum®. Product descriptions of Postum® state that the wheat has been roasted and mixed with a small amount of pure molasses. This composition has been used as a cereal food since the early twentieth century, and has been used as a coffee substitute beverage since the mid-twentieth century.

The invention discloses other methods of using this product apart from its nutritive and/or taste value.

### SUMMARY OF THE INVENTION

A method of suppressing appetite in an individual by ingesting an appetite-suppressant amount of wheat bran, wheat, molasses, and corn-derived maltodextrin at an interval prior to or with at least one meal to suppress appetite and thereby facilitate reduction of total caloric intake during the meal.

Another embodiment is a method of suppressing appetite in an individual by ingesting an appetite-suppressant amount of a grain such as wheat, corn, rye, rice, oats, or barley, that has been roasted for an interval sufficient to caramelize the grain, the ingestion with or at an interval prior to a meal to suppress appetite and thereby facilitate reduction of total caloric intake during the meal.

Another embodiment is a method of suppressing appetite in an individual by ingesting wheat bran, wheat, molasses and corn-derived maltodextrin with or at an interval prior to at least one meal to suppress appetite without the use of non-natural components, botanicals, or herbs.

Another embodiment is a kit comprising a composition of at least one of wheat bran, wheat, molasses and corn-derived maltodextrin, and instructions for consuming an appetite-suppressant amount of the composition with or at an interval prior to at least one meal to suppress hunger and thereby facilitate reduction of total caloric intake during the meal.

Another embodiment is a method of suppressing appetite in an individual by ingesting a foodstuff containing an appetite-suppressant amount of roasted wheat bran at an interval prior to or with at least one meal to suppress appetite and thereby facilitate reduction of total caloric intake during the meal. The foodstuff can also contain one or more of wheat, molasses, and corn-derived maltodextrin.

Another embodiment is a method of suppressing appetite in an individual by ingesting a beverage containing at least about one-half teaspoon of a composition comprising, in decreasing order, wheat bran, wheat, molasses, corn-derived maltodextrin, and natural coffee flavor, the composition dissolved in a liquid, at an interval prior to or with at least one meal to suppress appetite.

Another embodiment is a method of suppressing appetite in an individual by ingesting a beverage comprising an appetite-suppressant amount of at least one of wheat bran, wheat, molasses, corn-derived maltodextrin, and natural coffee flavor, the composition dissolved in a liquid, at an interval prior to or with at least one meal to suppress appetite.

The use of a mixture of roasted wheat bran that also contains wheat, molasses, corn-derived maltodextrin, and natural coffee flavor, commercially available as Postum®, for appetite suppression and/or weight reduction, is disclosed. Postum® is a coffee substitute and is formulated as a powder or crystals. It is conventionally prepared by dissolving in boiling or hot water, as one would prepare instant coffee. It may also be consumed in a non-heated or cold form after it is dissolved, or may be consumed as an ingredient in a food product.

In one embodiment, instructions are provided to consume Postum® before eating a meal (for example, up to one to two hours prior to eating) to suppress appetite. In another embodiment, instructions are provided to consume Postum® as a beverage during a meal to suppress appetite. Consumption of Postum® suppresses hunger and thereby facilitates reduction of total caloric intake during the meal, which can result in weight reduction.

In another embodiment, the invention also includes consuming a product containing wheat bran, wheat, corn, rye, oats, or another grain, that has been roasted to the point of carmelization. When the product is formulated as a beverage, an individual drinks a cup of this beverage as previously described to suppress appetite. When the product is formulated in a solid food, an individual consumes a serving of the food to suppress appetite.

Postum® is made from wheat and other natural components. There are no herbs, botanicals, or other synthetic components that may be harmful when consumed. There are no stimulants, such as caffeine. Thus, the inventive method of suppressing appetite and/or reducing weight is as safe as consuming Postum®, which has been available for over one hundred years.

These and other advantages will be apparent in light of the following drawing and detailed description.

### BRIEF DESCRIPTION OF THE DRAWING

FIG. 1 is a histogram showing weight loss of individuals practicing the inventive method.

### DETAILED DESCRIPTION

A method of desirably suppressing or reducing appetite is disclosed. In one embodiment, the invention includes consumption for appetite suppression of a natural coffee flavored foodstuff containing the following ingredients, either individually or collectively: wheat bran, wheat, molasses, corn-derived maltodextrin, where the grain may be roasted. In another embodiment, the invention includes consumption of a foodstuff containing an appetite-suppressant amount of wheat bran, either prior to a meal or concomitantly with a meal, to result in desirable appetite suppression. In another embodiment, the invention includes consumption for appetite suppression of a foodstuff containing a mixture of the following ingredients listed in relative decreasing order: wheat bran, wheat, molasses, corn-derived maltodextrin, and natural coffee flavor, commercially available as Postum®, prior to or with at least one meal to result in desirable appetite suppression.

A foodstuff is defined herein as a liquid (for example, a hot beverage, a shake, a smoothie, etc.), a solid (for example, snack bars, etc.), or a semi-solid (for example, sorbets, yogurts, etc.). The instruction may include foodstuff consumption at intervals throughout the day, or as a beverage with the meal.

A corollary effect of appetite suppression is weight reduction. Following weight loss, it is frequently desirable to maintain the reduced weight. In one embodiment, the invention also includes an instruction to consume a foodstuff containing an appetite-suppressant amount of wheat bran, either prior to a meal or concomitantly with a meal, to result in weight reduction and/or to maintain a desirable weight. In another embodiment, the invention also includes an instruction to consume a foodstuff containing, in relative decreasing order, a mixture of wheat bran, wheat, molasses, and corn-derived maltodextrin, commercially available as Postum®, at intervals throughout the day prior to eating a meal or with the meal to result in desirable weight reduction and/or to maintain a desirable weight. In another embodiment, the invention also includes an instruction to consume a foodstuff containing at least one of wheat bran, wheat, molasses, and corn-derived maltodextrin prior to a meal or with the meal to result in weight reduction and/or to maintain a desirable weight.

Postum® is available commercially from Kraft Foods, Rye Brook N.Y. and is sold in supermarkets. The label lists the ingredients as wheat bran, wheat, molasses, maltodextrin (from corn), and natural coffee flavor. The ingredients are listed in decreasing order, such that quantitatively the main ingredient is wheat bran.

Wheat bran is the rough outer covering of a wheat kernel, commonly known as a wheat berry. It adds flavor and fiber to baked goods. While not bound by a specific theory, bran is a known bulking agent and may account for a substantial degree of the appetite suppressant effects by increasing the sensation of satiety or fullness. This sensation may additionally or alternatively result from increasing the bulk in the stomach and upper digestive tract, and hence may actually increase the mass and/or volume of the stomach contents.

Wheat, molasses, maltodextrin (from corn), and natural coffee flavor may also account for and/or contribute to appetite-suppressant/weight reduction effects. Wheat flour results from processing the wheat endosperm, the majority of the wheat kernel or wheat berry, and containing starch, protein, niacin and iron. Molasses is the remaining brown-black liquid when juice is squeezed from sugar cane and sugar beets and boiled to a syrupy mixture from which sugar crystals are extracted. It is a sweetener and flavorant. Maltodextrin is also a sweetener and flavorant. It is a mixture of glucose, maltose, oligosaccharides, and polysaccharides obtained by partial hydrolysis of starch, and therefore also contains fiber. Natural coffee flavor is expected to provide flavorant properties (taste and/or aroma), and is not expected to contribute to the appetite suppressant effect.

The appetite suppressant amount of an ingredient will vary depending upon the properties of the ingredient, as known to one skilled in the art. As one example, wheat bran, which provides fiber, would be expected to have a greater appetite suppressant effect than wheat flour. As another example, wheat bran would be expected to contain a greater amount of fiber than maltodextrin, and thus would be expected to contribute more to an appetite suppressant effect than maltodextrin.

The ingredients may be present, individually or in combination, in various concentrations. The concentration of wheat bran may range from about 1%^{w/w} to about 20%^{w/w}. In one embodiment, the concentration of wheat bran ranges from about 5%^{w/w} to about 10%^{w/w} The concentration of wheat may range from about 5%^{w/w} to about 15%^{w/w}. In one embodiment, the concentration of wheat ranges from about 3%^{w/w} to about 5%^{w/w}. The concentration of molasses may range from about 0.2%^{w/w} to about 10%^{w/w}. In one embodiment, the concentration of molasses ranges from about 2%^{w/w} to about 5%^{w/w}. The concentration of maltodextrin may range from about 0.1%^{w/w} to about 5%^{w/w}. In one embodiment, the concentration of maltodextrin ranges from about 0.5%^{w/w} to about 2%^{w/w}. In one embodiment, up to about 35% of the composition is wheat and wheat bran. The grains may be roasted or unroasted.

Postum® is prepared according to package directions, typically, at least one teaspoonful, which is indicated on the label as about three grams, in about eight ounces of boiling water, and stirred until dissolved. It may also be prepared as one ingredient in solid foods such as chewy bars or semi-solid foods such as sorbet as listed on the manufacturer's web site (kraftfoods.com), or may be added to solid or semi-solid foods (for example, yogurt). The amount of Postum® may be increased, for example, one-and-one-half teaspoons, two teaspoonfuls, two-and-one-half teaspoonfuls, etc. The exact amount of Postum® may be determined by palatability, dissolvability in the volume of water typically contained in a coffee cup or mug, etc. While less than one teaspoonful of Postum® may be consumed in a serving, a lesser degree or extent of appetite suppression and/or rate of weight reduction may result. Flavoring agents such as milk, cream, non-dairy creamer, sugar, a sugar substitute, vanilla, or other flavorants may be added. The beverage or food is then consumed. If the beverage is hot or warm, it may be consumed incrementally in small sips, as one would consume hot or warm coffee. If the beverage is cold or chilled, it may be consumed in larger portions.

In one embodiment of the invention, the beverage or food is consumed prior to meals such that an appetite suppressant effect is achieved by the time the individual eats the meal. Consumption may occur anytime before a meal, for example, up to about two hours prior to eating, up to about one hour prior to eating, or immediately prior to eating. It may be consumed before each regular meal (that is, before breakfast, before lunch, and before dinner), or it may be consumed at specified meals (for example, before dinner only, etc.). In another embodiment, the beverage or food is consumed with the meal. The regimen may alternate these embodiments, for example, Postum® may be consumed prior to one meal, but concomitantly with another meal.

The individual experiences a feeling of satiety and thus is able to achieve reduced total caloric intake at each meal without feeling hungry. In one individual, drinking one cup of Postum® prior to each of breakfast, lunch, and dinner, reduced appetite to the extent that a weight loss of six pounds was achieved over a two-month period without a corresponding feeling of hunger and desire for food.

The following data were obtained from a group of twenty-one adults consuming Postum® according to the inventive method. More specifically, ten men and eleven women were asked to prepare and consume Postum® as a hot beverage before or during at least one of breakfast, lunch, or dinner. They were instructed to consume meals that did not qualitatively differ from their meals prior to the study; that is, the content of their meals did not change. They were also instructed to continue any on-going exercise regimen, but not to initiate a new exercise regimen.

None of the twenty-one participants was able to drink Postum® before each meal; that is, before breakfast, before lunch, and before dinner. However, all participants reported that they consumed the beverage before at least 50% of their meals. The compliance range was between 50% and 80% of the meals for all participants.

The data are as follows.

| | Weight Change | | Weight Change |
|---|---|---|---|
| Participant | (pounds) | Participant | (pounds) |
| Male 1 | −5 | Female 1 | −2.6 |
| Male 2 | −4 | Female 2 | −5 |
| Male 3 | 0 | Female 3 | +1.8 |
| Male 4 | −5 | Female 4 | +0.5 |
| Male 5 | −4 | Female 5 | +3 |
| Male 6 | −4 | Female 6 | −2 |
| Male 7 | −3 | Female 7 | +0.5 |
| Male 8 | −2 | Female 8 | +6 |
| Male 9 | 0 | Female 9 | 0 |
| Male 10 | −3 | Female 10 | 0 |
| | | Female 11 | −2 |

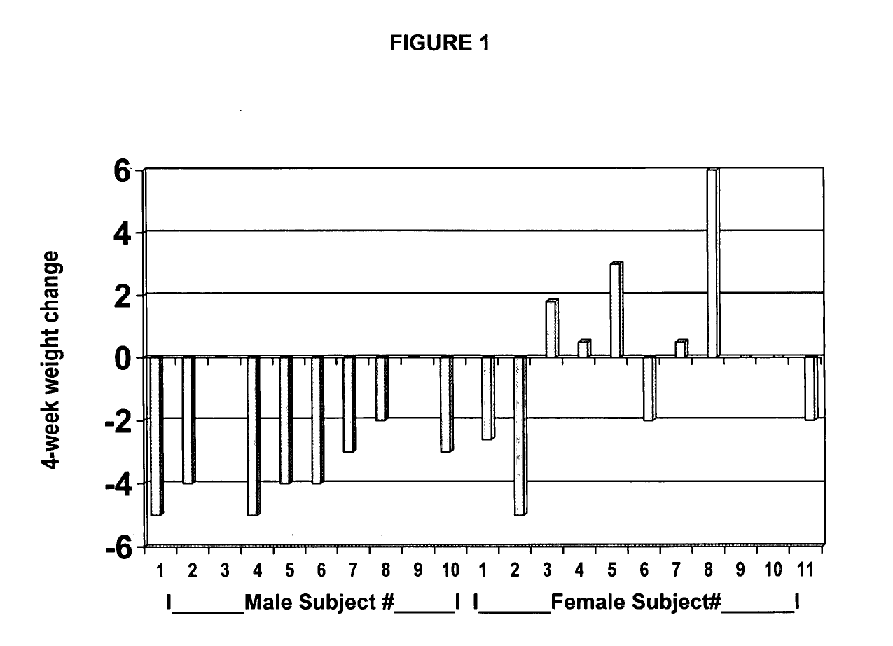

Comparative results are shown in FIG. 1. As illustrated in FIG. 1, none of the male participants gained weight. Eight of the ten male participants lost between two pounds and five pounds over the four-week period. The mean weight change for males over the four-week period was a weight loss of three pounds.

In contrast, five of the female participants gained weight. Four of the females lost weight during the four-week period, and two of the females maintained their weight. Of the five females who gained weight, two of these gained only half a pound, one gained almost two pounds, one gained three pounds, and the other gained six pounds. The mean weight change for females over the four-week period was a weight gain of 0.2 pounds.

The weight loss results for male participants compared to female participants was statistically significant (p≦0.05). Specifically, the male participants had a mean weight change of −3.0 pounds ±−0.58 pound. The female participants had a mean weight change of +0.018 pound ±0.897 pound. Thus the weight loss experienced by men was significant at the 95% confidence interval.

All of the participants, both male and female, reported ease of compliance with the study regimen. All of the participants who lost weight (eight men and four women) decided to continue with the regimen on their own. All of these individuals reported that they are pleased with their “effortless” loss of weight.

While not wishing to be bound by any theory, the male and female participants who reported no weight loss, or reported a weight gain, were questioned about their eating habits between meals. All of the women who gained weight reported snacking between meals, and one of the two men who did not experience a weight change also reported snacking between meals.

It will be appreciated that the components or ingredients in Postum®, that is, grains and grain products, are natural. Postum® also lacks the stimulant caffeine that is found in coffee. Additionally, wheat bran, wheat, molasses, and corn-derived maltodextrin are well-characterized and known components. The method thus achieves appetite suppression and/or weight reduction and/or weight maintenance with natural components whose properties are well known. That is, it achieves these effects without the use of botanicals, herbs, nutriceuticals, and other components that, while found in nature, may have effects that are potentially or actually harmful. Thus, the invention provides a method of appetite suppression and/or weight reduction that is achieved without either synthetic compounds, or without compounds that may have potentially harmful effects due to uncharacterized components or interactions.

In another embodiment, consumption in liquid or solid form of roasted grain may be used as an appetite suppressant, and instructions for consumption to achieve weight reduction may be provided. Grains, such as wheat, corn, rice, rye, barley, oats, malt, etc., undergo a carmelization process when they are roasted due to slow heating of the natural sugars in these grains. Carmelization is a transformational process of sugar(s) in the foodstuff brought about by frictional heating of the foodstuff at a sufficiently rapid rate so that an exothermic reaction degrades the sugar(s). Glycosidic bonds degrade, followed by condensation and the formation of water, then carmelization begins. The heating rate is controlled to avoid burning the foodstuff by too rapid heating, and to avoid sweating the foodstuff by too slow heating. Once carmelization begins, the foodstuff must not exotherm, or lose heat, which will impart a baked taste. Carmelization may impart flavor, mouthfeel, texture, and other properties to foods. Thus, the roasting process and carbonization and carmelization reactions may also contribute to the sensation or condition of satiety. The invention also comprises instructions for consuming a grain product that has been roasted to impart carmelization to the product for the purpose of appetite suppression and/or weight reduction.

It will be appreciated that the mechanism by which Postum® suppresses appetite, and hence reduces food consumption, is unnecessary to practice the inventive method.

Other variations or embodiments of the invention will also be apparent to one of ordinary skill in the art from the above description. As one example, instructions may indicate intermittent consumption of the beverage or food to assist an individual in maintaining a desired weight. As another example, instructions may indicate consumption of the beverage or food as part of a health and/or diet regimen. Thus, the forgoing embodiments are not to be construed as limiting the scope of this invention.

1. A method of suppressing appetite in an individual comprising ingesting an appetite-suppressant amount of wheat bran, wheat, molasses, and corn-derived maltodextrin at an interval prior to or with at least one meal to suppress appetite and thereby facilitate reduction of total caloric intake during the meal.

2. The method of claim 1 wherein ingestion is up to about two hours prior to eating the meal.

3. The method of claim 1 wherein ingestion is of a foodstuff.

4. The method of claim 1 wherein ingestion is at regular intervals.

5. The method of claim 1 wherein ingestion is part of a weight reduction system.

6. The method of claim 1 wherein at least one of the wheat bran and wheat is roasted.

7. A method of suppressing appetite in an individual comprising ingesting an appetite-suppressant amount of a grain that has been roasted for an interval sufficient to carmelize the grain, the ingestion with or at an interval prior to a meal to suppress appetite and thereby facilitate reduction of total caloric intake during the meal.

8. The method of claim 7 wherein the roasted grain is at least one of wheat, wheat bran, corn, rye, rice, oats, and barley.

9. The method of claim 7 wherein ingestion is from about two hours prior to eating the meal.

10. The method of claim 7 wherein ingestion is of a foodstuff.

11. The method of claim 7 wherein ingestion is at regular intervals.

12. The method of claim 7 wherein ingestion is part of a weight reduction regimen.

13. A method of suppressing appetite in an individual comprising ingesting wheat bran, wheat, molasses, and corn-derived maltodextrin with or at an interval prior to at least one meal to suppress appetite without the use of non-natural components, botanicals, or herbs.

14. The method of claim 13 further without the use of a stimulant.

15. The method of claim 13 wherein ingestion is of a foodstuff.

16. The method of claim 13 wherein at least one of the wheat bran and wheat is roasted.

17. A kit comprising a composition of wheat bran, wheat, molasses, and corn-derived maltodextrin, and instructions for consuming an appetite-suppressant amount of the composition with or at an interval prior to at least one meal to suppress hunger and thereby facilitate reduction of total caloric intake during the meal.

18. The kit of claim 17 where the instructions are provided for weight reduction.

19. The kit of claim 17 wherein the instructions provide for consuming the composition as at least one of a liquid or a solid.

20. A method of suppressing appetite in an individual comprising ingesting a foodstuff comprising an appetite-suppressant amount of wheat bran at an interval prior to or with at least one meal to suppress appetite and thereby facilitate reduction of total caloric intake during the meal.

21. The method of claim 20 wherein the foodstuff further comprises wheat, molasses, and corn-derived maltodextrin.

22. The method of claim 20 wherein the foodstuff is ingested from about two hours prior to eating the meal.

23. The method of claim 20 wherein the foodstuff is ingested at regular intervals.

24. The method of claim 20 wherein the foodstuff is ingested as part of a weight reduction system.

25. The method of claim 20 wherein at least one of the wheat bran and wheat is roasted.

26. A method of suppressing appetite in an individual comprising ingesting a beverage comprising at least about one-half teaspoon of a composition comprising, in decreasing order, wheat bran, wheat, molasses, corn-derived maltodextrin, and natural coffee flavor, the composition dissolved in a liquid, at an interval prior to or with at least one meal to suppress appetite.

27. A method of suppressing appetite in an individual comprising ingesting a beverage comprising an appetite-suppressant amount of at least one of wheat bran, wheat, molasses, corn-derived maltodextrin, and natural coffee flavor, the composition dissolved in a liquid, at an interval prior to or with at least one meal to suppress appetite.

28. The method of claim 27 wherein at least one of the wheat bran or wheat is roasted.

29. The method of claim 27 wherein the composition comprises from about 1 %^{w/w} wheat bran to about 20%^{w/w} wheat bran.

30. The method of claim 27 wherein the composition comprises from about 5%^{w/w} wheat bran to about 10%^{w/w} wheat bran.

31. The method of claim 27 wherein the composition comprises from about 5%^{w/w} wheat to about 15%^{w/w} wheat.

32. The method of claim 27 wherein the composition comprises from about 3%^{w/w} wheat to about 5%^{w/w} wheat.

33. The method of claim 27 wherein the composition comprises from about 0.2%^{w/w} molasses to about 10%^{w/w} molasses.

34. The composition of claim 27 wherein the composition comprises from about 2%^{w/w} molasses to about 5%^{w/w} molasses

35. The method of claim 27 wherein the composition comprises from about 0.1%^{w/w} maltodextrin to about 5%^{w/w} maltodextrin.

36. The composition of claim 27 wherein the composition comprises from about 0.5%^{w/w} maltodextrin to about 2%^{w/w} maltodextrin.
